(19) United States
(12) Patent Application Publication
(10) Pub. No.: US 2025/0050188 A1
(43) Pub. Date: Feb. 13, 2025

(54) SYSTEMS AND METHODS FOR PREDICTIVE SHOULDER KINEMATICS OF REHABILITATION EXERCISES THROUGH IMMERSIVE VIRTUAL REALITY

(71) Applicant: The Regents of the University of California, Oakland, CA (US)

(72) Inventors: Michael Powell, Oakland, CA (US); Aviv Elor, Santa Cruz, CA (US); Ash Robbins, Oakland, CA (US); Mircea Teodorescu, Santa Cruz, CA (US); Sri Kurniawan, Santa Cruz, CA (US)

(21) Appl. No.: 18/717,978

(22) PCT Filed: Dec. 8, 2022
(86) PCT No.: PCT/US2022/081202

§ 371 Date: Jun. 7, 2024

Related U.S. Application Data
(60) Provisional application No. 63/265,145, filed on Dec. 8, 2021.

(51) Int. Cl. A63B 71/06; A63B 24/00; G06F 3/01; G06F 30/27; G06T 7/00; G06T 7/246; G06T 7/292; G06T 19/20; G16H 20/30

(52) U.S. Cl. CPC A63B 71/0622; A63B 24/0062; G06F 30/27; G06T 7/0012; G06T 7/251; G06T 7/292; G06T 19/20; G16H 20/30; A63B 2071/0638; A63B 2071/0666; A63B 2220/24; A63B 2220/54; A63B 2220/805; A63B 2220/836; G06F 3/011; G06T 2207/10048; G06T 2207/20016; G06T 2207/20081; G06T 2207/20084; G06T 2207/30008; G06T 2210/41; G06T 2219/024; G06T 2219/2016

(57) ABSTRACT

Methods and systems are provided for predictive shoulder kinematics via immersive virtual reality. In one example, a system comprises an immersive virtual reality (iVR) system, the iVR system including a headset and a hand-held controller, and machine readable instructions executable to: predict joint kinematics using a machine learning model based on motion data received from the iVR system during gameplay of a virtual reality-guided exercise with the iVR system. In this way, physical rehabilitation may be performed remotely with increased evaluation accuracy.

### CROSS-REFERENCE TO RELATED APPLICATION

The present application claims priority to U.S. Provisional Application No. 63/265,145 entitled “SYSTEMS AND METHODS FOR PREDICTIVE SHOULDER KINEMATICS OF REHABILITATION EXERCISES THROUGH IMMERSIVE VIRTUAL REALITY”, and filed on Dec. 8, 2021. The entire contents of the above-listed application is hereby incorporated by reference for all purposes.

### ACKNOWLEDGMENT OF GOVERNMENT SUPPORT

This invention was made with Government support under Grant No. 1521532, awarded by the National Science Foundation. The Government has certain rights in the invention.

### FIELD

The present description relates generally to telehealth-mediated physical rehabilitation.

### BACKGROUND/SUMMARY

The adoption of telehealth rapidly accelerated due to the global COVID19 pandemic disrupting communities and in-person healthcare practices. Telehealth also helps make healthcare more equitable by helping patients overcome obstacles related to geography, time, finances, and access to technology. Moreover, telehealth has been found to be effective in musculoskeletal practices, having demonstrated outcomes and patient satisfaction comparable to in-person care. While telehealth had initial benefits in enhancing accessibility for remote treatment, physical rehabilitation has been heavily limited due to the loss of hands-on evaluation tools. Immersive virtual reality (iVR) offers an alternative medium to video conferencing. Stand-alone head-mounted display systems are becoming more affordable, user friendly, and accessible to many users at once. Further, such virtual experiences can be built with privacy protocols that satisfy healthcare regulations. The systems use low-cost motion tracking methods to match user movement in the real world to that in the virtual environment. An advantage iVR offers over videoconferencing is the ability for patients and therapists to meet in a three-dimensional, interactive virtual environment.

However, metrics for remote evaluation using iVR have not yet been established. Further, upper limb kinematics, particularly of the shoulder joint, may be difficult to evaluate. For example, the structure of the shoulder allows for tri-planar movement that cannot be estimated by simple single plane joint models.

In one example, the issues described above may be at least partially addressed by a system, comprising: an immersive virtual reality (iVR) system, the iVR system including a headset and a hand-held controller, and machine readable instructions executable to: predict joint kinematics using a machine learning model based on motion data received from the iVR system during gameplay of a virtual reality-guided exercise with the iVR system. In this way, remote physical rehabilitation may be provided with clinically meaningful evaluation metrics.

As one example, the machine learning model may be trained using joint angles and joint torques determined via biomechanical simulation from data obtained via an optical motion tracking system. For example, the optical motion tracking system may comprise a plurality of reflective markers positioned at anatomical landmarks and a plurality of cameras that may track positions of the reflective markers over time. Further, the machine learning model may comprise a plurality of separate models for different parameters of the joint kinematics. The plurality of separate models for the different parameters of the joint kinematics may comprise an elevation plane angle model, an elevation angle model, an elevation plane torque model, an elevation torque model, and a rotation torque model, for example. As another example, the machine learning model may be trained using an extreme gradient boost algorithm, an artificial neural network, a convolutional neural network, a long short-term memory, and/or a random forest. As a result, the system may accurately predict the joint kinematics using the low-cost iVR system, thus increasing patient access to clinically meaningful remote physical rehabilitation.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 shows an overview of using an immersive virtual reality system for predictive shoulder kinematics.

FIG. 2 shows a schematic overview of a system for developing a machine learning model for predicting joint angles and torques using a virtual reality-guided exercise.

FIG. 3 shows movements that may be included in the virtual reality-guided exercise for shoulder rehabilitation.

FIG. 4 shows a set of loss function graphs for training machine learning models of shoulder parameters.

FIG. 5 shows a set of graphs illustrating vertical displacement of a hand-held controller during a plurality of different guided movements.

FIG. 6 shows graphs comparing shoulder elevation angles and torques determined using an offline biomechanical simulation method versus a machine learning-based method.

### DETAILED DESCRIPTION

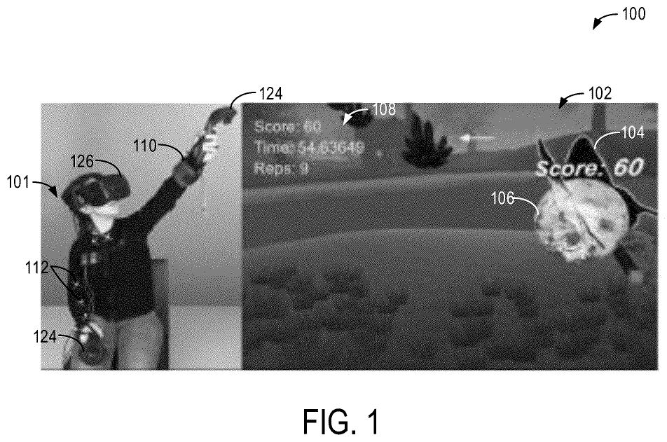

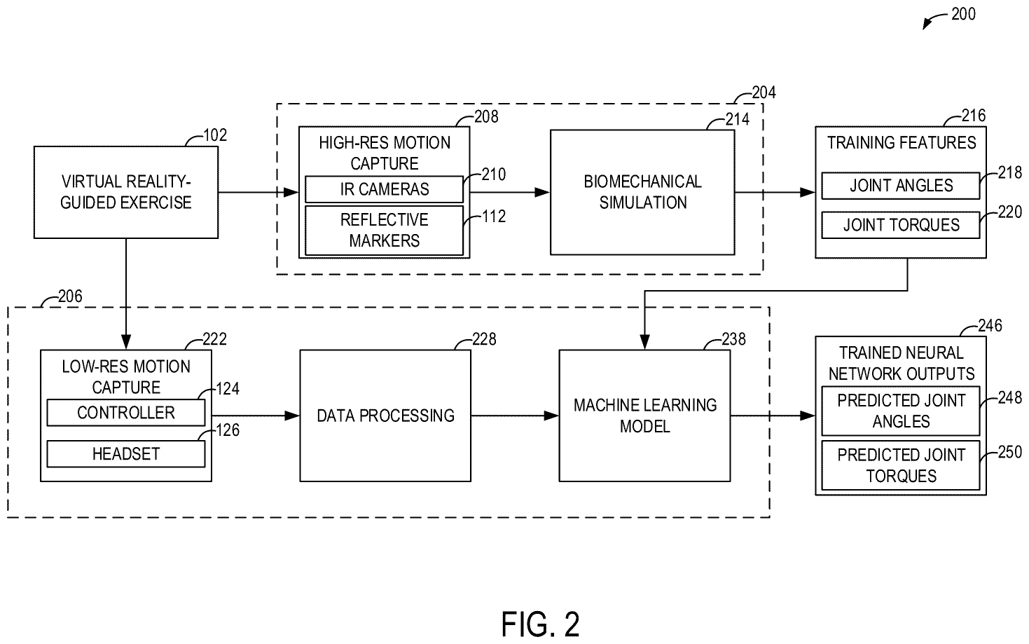

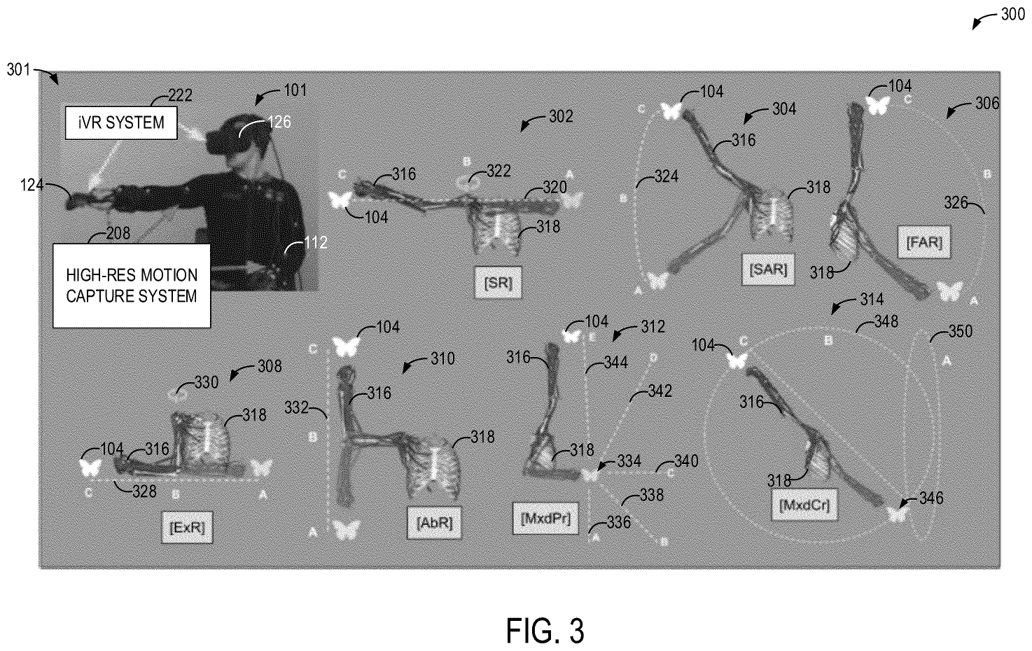

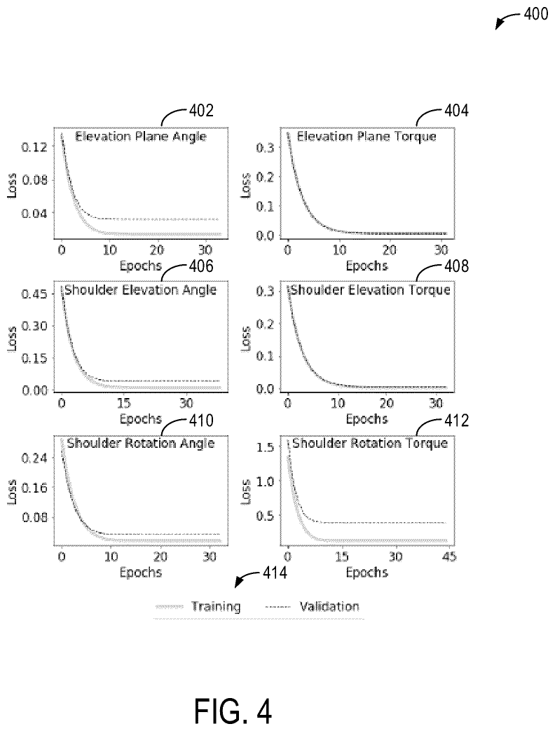

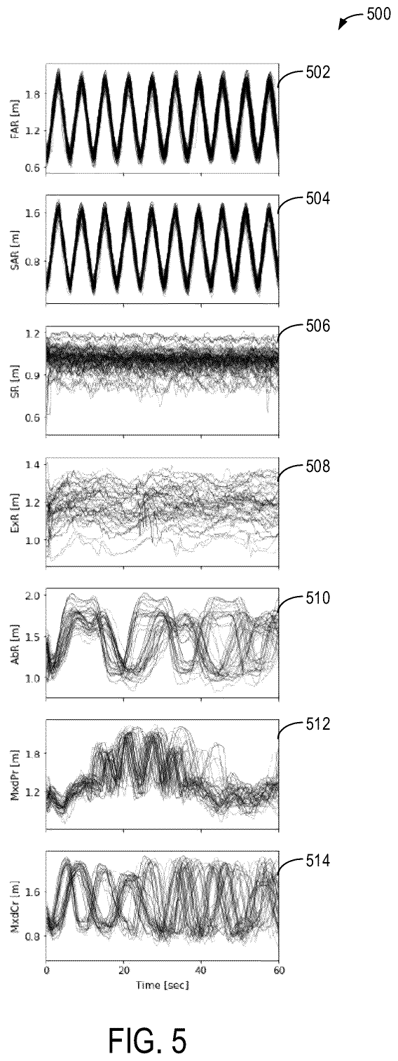

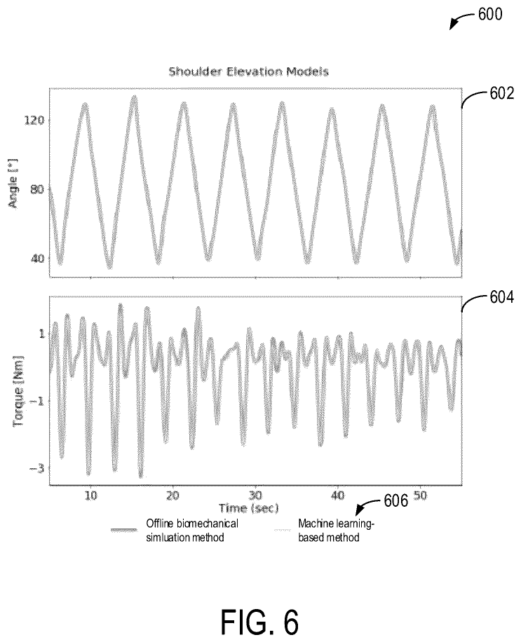

The following description relates to systems and methods for predictive shoulder kinematics via immersive virtual reality (iVR), such as using the system shown in FIG. 1. For example, the predictive shoulder kinematics may be determined via a machine learning model that is trained using biomechanical simulation data generated from high-resolution motion capture data, such as according to the workflow of FIG. 2. Both the high-resolution motion capture data and data from the iVR system may be obtained during an iVR game that guides a user through a plurality of movements selected to aid rehabilitation, such as the movements shown in FIG. 3. The training may result in a plurality of different models that can be optimized using a loss function, such as illustrated in FIG. 4. Further, the plurality of movements may result in varying uniformity across gameplays, as illustrated in FIG. 5. However, even with the varying inputs, the trained models accurately replicate the output of the biomechanical simulation, as is illustrated in FIG. 6. As a result, the trained machine learning model provides accurate predictions of shoulder torques and angles, that can be used by a therapist to evaluate shoulder rehabilitation, using only data from the iVR system.

Turning now to the figures, FIG. 1 shows an overview 100 of using an immersive virtual reality (iVR) system for determining predictive shoulder kinematics via a virtual reality-guided exercise 102. A user (or subject) 101 wears a plurality of reflective markers 112 for a high-resolution motion tracking system in addition to a headset 126 and hand-held controllers 124 of a low-resolution motion tracking system, as will be elaborated below with respect to FIG. 2. The user 101 may exercise one arm at a time wearing a weighted arm strap 110.

The virtual reality-guided exercise 102 is a game that guides the user through a series of movements selected to aid rehabilitation, as will be elaborated upon below with respect to FIGS. 2 and 3. Shoulder rehabilitation is described in the present example, although the systems and methods described herein may be similarly applied to other joints by one of skill in the art in light of this disclosure. In the example shown in FIG. 1, the virtual reality-guided exercise 102 includes the user 101 protecting a moving butterfly 104 with an orb 106. For example, the butterfly 104 may move across a display screen, as shown via the headset 126, according to the series of movements, and the user 101 may move the hand-held controller 124 to place the orb 106 on the butterfly 104. Correctly placing the orb 106 on the butterfly 104, and thus protecting the butterfly 104, scores points, which may be displayed via a tabulation 108 that also shows a duration of the virtual reality-guided exercise 102, a number of reps performed for a given movement, etc. The motion of the user 101 is tracked via both the high-resolution motion tracking system and the low-resolution motion tracking system. As will be elaborated below with respect to FIG. 2, data from the high-resolution motion tracking system may be used to build a machine learning model able to analyze data from the low-resolution motion tracking system. Further, once the machine learning model is trained, the machine learning model may be supplied with data from the low-resolution motion tracking system alone to predict shoulder kinematics.

FIG. 2 shows a block diagram of an exemplary system 200 for developing a machine learning model for predictive shoulder kinematics. Components of FIG. 2 that function the same as those introduced in FIG. 1 are numbered the same and will not be reintroduced. The system 200 includes the virtual reality-guided exercise 102, and data obtained from the virtual reality-guided exercise 102 is processed via both an offline processing method 204 and a machine learning-based processing method 206. The offline processing method 204, also referred to herein as a traditional processing method for biomechanical simulations, utilizes a high-resolution motion capture system 208. The high-resolution motion capture system 208 includes a plurality of infrared cameras 210 that receive data from the plurality of reflective markers 112 positioned on a user (e.g., the user 101 of FIG. 1) at defined locations to capture the user's movements in a technique referred to as optical motion capture. The plurality of infrared cameras 210 may capture images at a high frame rate, such as a frame rate of 120-240 frames per second (FPS). As one example, the plurality of infrared cameras 210 may be an off-the-shelf infrared camera such as an infrared camera manufactured by OptiTrack. As an example, at least eight infrared cameras 210 may be used and radially distributed about the subject in order to track movement of the plurality of reflective markers 112 within three-dimensional (3D) world space, including a position (e.g., in x, y, z coordinates) of each marker. Further, the plurality of reflective markers 112 may include at least ten reflective markers placed on bony landmarks of the user, particularly on the arm and shoulder.

In contrast, the machine learning-based processing method 206 includes a low-resolution motion capture system 222 that further includes the one or more hand-held controllers 124 and the headset 126. The low-resolution motion capture system 222 also may be referred to herein as an iVR system 222. The low-resolution motion capture system 222 may be an off-the-shelf iVR system, such as the HTC Vive. For example, the low-resolution motion capture system 222 may use one or more sensors to track a position and rotation (e.g., angles with respect to the x, y, and z axes, referred to as roll, pitch, and yaw) of the one or more hand-held controllers 124 in 3D world space. Similarly, one or more sensors may track a position of the headset 126 in 3D world space, which may affect an image shown to a user via the headset 126. For example, the headset 126 may include an immersive display, and as the user moves their head and changes the position of the headset 126 in 3D word space, the image shown on the immersive display may change accordingly. Further, an indicator of the position of the one or more hand-held controllers 124 may also be shown on the immersive display, such as the orb 106 described above with respect to FIG. 1.

As elaborated herein, the virtual reality-guided exercise 102 guides the user through rehabilitation-relevant movements via the immersive display of the headset 126. The high-resolution motion capture system 208 and the low-resolution motion capture system 222 both track the motions of the user as the user performs the movements. Data from the high-resolution motion capture system 208 is processed via a biomechanical simulation 214 that outputs training features 216 for joint parameters, including joint angles 218 and joint torques 220. The training features 216 may be input into a machine learning model 238 of the machine learning-based processing method 206, as will be elaborated below. Shoulder joint parameters will be described herein, although the system 200 could be similarly used to determine parameters for other joints. For example, the virtual-reality guided exercise 102 may guide the user through shoulder rotation (SR), side arm raise (SAR), forward arm raise (FAR), external rotation (ExR), abducted rotation (AbR), mixed press (MxdPr), and mixed circles (MxdCr) movements, as will be further described with respect to FIG. 3.

The high-resolution motion capture system 208 is considered the gold standard for accuracy and precision in motion tracking but is often restricted to laboratory environments due to its size and expense. Therefore, data from the high-resolution motion capture system 208 may be used to generate accurate training data via the biomechanical simulation 214. To collect the training data, positions of the plurality of reflective markers 112, as captured by the IR cameras 210, are used as inputs into the biomechanical simulation 214 for inverse kinematics. For example, the biomechanical simulation 214 may be an inverse kinematics tool of OpenSim software that incorporates an upper body model. The biomechanical simulation 214 positions the model to best fit the data from the plurality of reflective markers 112 at each time frame, such as by finding the model pose that minimizes the sum of weighted squared errors of the markers, as shown in

$$Equation 1$$

$$SE = {\sum }_{i ∈ m} {w}_{i} {‖ {x}_{i}^{exp} - {x}_{i} ‖}^{2} + {\sum }_{j ∈ uc} {{w}_{j} ( {q}_{j}^{exp} - {q}_{j} )}^{2} \qquad ( 2 )$$

where SE is the squared error, m are the plurality of reflective markers 112, uc are a set of unprescribed coordinates, xᵢ^{exp} is the experimental position of marker i, xᵢ is the position of the corresponding model marker, qᵢ^{exp} is the experimental value for coordinate j, wᵢ are the marker weights, w; are the coordinate weights, and q=qⱼ^{exp} for all prescribed coordinates j.

Inverse dynamics may be used to determine net forces and torques at each joint (e.g., the joint torques 220). For example, the inverse dynamics may be a tool within the biomechanical simulation 214 that uses results from the inverse kinematics tool and external loads applied to the model using classical equations of motion, such as Equation 2:

$$M ( q ) \ddot{q} + C ( q , \dot{q} ) + G ( q ) = τ \qquad ( 2 )$$

where q, \dot{q}, \ddot{q} ∈ [symbol] ^{N} are the vectors of generalized position, velocities, and accelerations, respectively; M(q)∈ [symbol] ^{NxN} is the system mass matrix; C(q, \dot{q})∈ [symbol] ^{N} is the vector of Coriolis and centrifugal forces; G(q)∈ [symbol] ^{N} is the vector of gravitational forces; and π∈ [symbol] ^{N} is the vector of generalized forces. The model's motion is defined by the generalized positions, velocities, and accelerations to solve for a vector of generalized forces.

In the example shown in FIG. 1, the user is seated during gameplay of the virtual reality-guided exercise 102 so that there is little movement of the torso or head. As a result, the headset 126 moves very little. In the example shown in FIG. 1, only one arm may be used during gameplay. Therefore, in the example shown in FIG. 1, only one of the hand-held controllers 124 may substantially provide input in determining the joint mechanics and dynamics of the moving arm. Thus, data from the hand-held controller 124 used during gameplay, including the x, y, and z positions along with the roll, pitch, and yaw rotation of the moving controller, may undergo data processing 128. Further, any weight applied to the arm (e.g., via the weighted arm strap 110 of FIG. 1) may also be input into the data processing 228.

The data processing 228 may include data cleaning, feature selection, interpolation, and batch generation. The low-resolution motion capture system 222 and the high-resolution motion capture system 208 collect data at different frequencies, and thus interpolation is used to synchronize data to a common timeline. The collected data is scanned for any outliers or missing values so that it may be corrected if any are detected. The data is cropped into smaller segments, which are later randomized for training the machine learning model 238. This randomization provides more generalizable results rather than training on single data set that is in chronological order.

An illustrative example of the data processing 228 and model building via the machine learning model 238 will now be described. The illustrative example uses 540 game trials of the virtual reality-guided exercise 102, each recorded for 60 seconds at 120 Hz to generate a data set of approximately 3.89 million instances (e.g., arm positions). A set of 54 (10%) randomly selected trials are selected as a test set to test the final models. The remaining 60 second recordings are split into segments of 3 seconds. These shorter segments may be used to prevent the model from learning patterns in the movements due to the repetitive nature of some of the movements. Each segment is randomly placed into the training or validation set such that the overall data is split into 80% training (e.g., using data produced via the offline processing method 204), 10% validation, and 10% test.

There are many types of machine learning models available that each use different types of data and prediction methods. Typically, these machine learning models perform regression, clustering, visualization, or classification and can use probabilistic methods, rule-based learners, linear models (e.g., neural networks or support vector machines), decision trees, instance-based learners, or a combination of these. The type of input data may be taken into consideration to select the approach used for the machine learning model 238 in order to determine what type of prediction is needed (e.g., binary classification, multiclass classification, regression, etc.), identify the types of models that are available, and consider the pros and cons of those models. Examples of elements to consider are accuracy, interpretability, complexity, scalability, time to train and test, prediction time after training, and generalizability.

In the present example, the machine learning model 238 uses gradient boosting and a decision tree to perform a supervised multiple regression task because there are multiple input variables and the input and output data are already known and numeric. The decision tree 242 includes a simple predictive model including bagging, random forest, boosting, and gradient boosting. Extreme Gradient Boosting (XGBoost) builds upon all of these methods to increase speed and performance. XGBoost may be used because of its ability to accurately train on the specific type of input data as well as its built in regularization methods (e.g., LASSO and Ridge) to ensure the machine learning model 238 does not over-fit the data. Alternatively, other algorithms may be used, such as Artificial Neural Networks (ANNs), Convolutional Neural Networks (CNNs), Long Short-Term Memory (LSTM), and Random Forests. In the present example, the machine learning model 238 may comprise six models to produce joint and torque predictions, as specified in Table 1 below.

TABLE 1

| Model Inputs (N = 7) | Model Outputs (N = 6) |
|---|---|
| Controller Position_x (m) | Elevation Plane Angle (°) |
| Controller Position_y (m) | Shoulder Elevation Angle (°) |
| Controller Position_z (m) | Shoulder Rotation Angle (°) |
| Controller Rotation_x (°) | Elevation Plane Torque (Nm) |
| Controller Rotation_y (°) | Shoulder Elevation Torque (Nm) |
| Controller Rotation_z (°) | Shoulder Rotation Torque (Nm) |
| Arm Strap Weight (kg) | |

Shoulder elevation describes rotation about the horizontal axis of the glenohumeral joint, elevation plane describes rotation about the vertical axis of the glenohumeral joint, and shoulder rotation describes rotation about the longitudinal axis of the humerus. Data from the biomechanical simulation 214 may be interpolated to match the collection frequency of the low-resolution motion capture system 222. The number of estimators may be set to 5,000, and the max depth may be set to 10 as values higher than this may provide little if any improvement. To prevent overfitting, early stopping rounds may be used for each model. As such, the training may stop and use the model of best fit (e.g., as determined via a loss function) if the model does not improve within five epochs. The validation data may be used after each epoch of training to determine if the training should be stopped due to no more improvement (known as early stopping). The training and validation will be further described with respect to FIG. 4.

Continuing with FIG. 2, once developed, the machine learning model 238 may be used to predict outputs from the unseen test set as trained neural network outputs 246. Further, the trained neural network outputs 246 may be filtered using a third order low-pass Butterworth filter with a cutoff frequency of 3 Hz to remove noise from the signal that is not attributed to the user's movement. The trained neural network outputs 246 include predicted joint parameters, including predicted joint angles 248 and predicted joint torques 250. That is, the predicted joint angles 248 and the predicted joint torques 250 may be determined using the machine learning model 238, which is trained via data derived from the high-resolution motion capture system 208.

The model may be evaluated using mean absolute error (MAE) to compare each model's prediction to the results from the biomechanical simulation 214 for the unseen test set, such as by using Equation 3:

$$MAE = \frac{{\sum }_{i = 1}^{n} \vert {y}_{i} - {x}_{i} \vert}{n} \qquad ( 3 )$$

where n is the number of data points, y is the prediction of the model, and x is the value obtained from the biomechanical simulation 214. Unlike the validation data, the test set is not processed by the machine learning model 238 until the training is complete. Instead, the test data is used to check how accurately the trained machine learning model 238 predicts on unseen data, such as using the MAE approach described above.

For example, the data from the high-resolution motion capture system 208 in the unseen test data set may be used to determine joint angles and torques in the biomechanical simulation 214. The averages and standard deviations of joint angles and joint torques of the biomechanical simulation 214 can be seen in Table 2 below. The MAE comparing the results from the biomechanical simulation 214 and the machine learning model 238 for the unseen test data set is shown in Table 3. As an example, the trained machine learning model 238 may generate predictions in runtime at an average rate of approximately 0.74 milliseconds (ms) for a single instance of inputs, making the machine learning model 238 both quick and highly accurate. For example, as shown in Table 3 below, the MAE was found to be less than 0.78 degrees for joint angles and less than 2.34 Nm for joint torques, indicating that the motion of the iVR system 222 provides enough input for accurate prediction using the machine learning model 238. Specifically, the rotation and position of the hand-held controller 124, along with the trained arm's wrist weight (e.g., from the weighted arm strap 110 of FIG. 1), are the only metrics input into the trained machine learning model 238.

TABLE 2

| | Biomechanical | Biomechanical Simulation |
|---|---|---|
| Motion | Simulation Angle (°) | Torque (Nm) |
| Elevation Plane | 76.9 ± 30.3 | 0.88 ± 1.62 |
| Shoulder Elevation | −23.8 ± 48.6 | 9.16 ± 2.37 |
| Shoulder Rotation | 35.8 ± 28.5 | 101.9 ± 246.9 |

TABLE 3

| Motion | Kinematics MAE (°) | Dynamics MAE (Nm) |
|---|---|---|
| Elevation Plane | 0.78 | 0.06 |
| Shoulder Elevation | 0.65 | 0.07 |
| Shoulder Rotation | 0.43 | 2.34 |

Thus, once trained and validated, the machine learning model 238 may be used to predict joint angles and torques of a subject wearing only the low-resolution capture system 222 and without additional input from the offline processing method 204.

Further, it may be understood that each of the offline processing method 204 and the machine learning-based processing method 206 may be executed by one or more processors operatively coupled to one or more memories (e.g., a tangible and non-transient computer readable medium). As used herein, the term “tangible computer readable medium” is defined to include any type of computer readable storage and to exclude propagating signals. Additionally or alternatively, the example methods and systems may be implemented using coded instruction (e.g., computer readable instructions) stored on a non-transitory computer readable medium such as a flash memory, a read-only memory (ROM), a random-access memory (RAM), a cache, or any other storage media in which information is stored for any duration (e.g., for extended time periods, permanently, brief instances, for temporarily buffering, and/or for caching of the information). Memory and processors as referred to herein can be standalone or integrally constructed as part of various programmable devices, including for example, computers or servers. Computer memory of computer readable storage mediums as referenced herein may include volatile and non-volatile or removable and non-removable media for a storage of electronic-formatted information, such as computer readable program instructions or modules of computer readable program instructions, data, etc. that may be stand-alone or as part of a computing device. Examples of computer memory may include (but are not limited to) RAM, ROM, EEPROM, flash memory, CD-ROM, DVD-ROM or other optical storage, magnetic cassettes, magnetic tape, magnetic disc, or other magnetic storage devices, or any other medium which can be used to store the desired electronic format of information and which can be accessed by the processor or processors or at least a portion of a computing device.

Further still, one or both of the offline processing method 204 and the machine learning-based processing method 206 may be implemented by one or more networked processors or computing devices. Such communicative connections may include, but are not limited to, a wide area network (WAN); a local area network (LAN); the internet; a wired or wireless (e.g., optical, Bluetooth, radio frequency) network; a cloud-based computer infrastructure of computers, routers, servers, gateways, etc.; or any combination thereof associated therewith that allows the system or portions thereof to communicate with one or more computing devices. As an illustrative example, data acquired by the low-resolution motion capture system 222 may be wirelessly transmitted to one or more computing devices, and the one or more computing devices may perform the data processing 228 and/or input the processed data into the machine learning model 238.

Turning now to FIG. 3, a plurality of virtual reality-guided movements 300 are shown. As described above, a virtual reality-guided exercise (e.g., the virtual reality-guided exercise 102 of FIGS. 1 and 2) may guide a user through a series of movements aimed at shoulder rehabilitation. Components of FIG. 3 that function the same as those described in FIGS. 1 and 2 are numbered the same and will not be reintroduced. For example, a system overview 301 that summarizes the systems described with respect to FIGS. 1 and 2 is shown alongside the movements 300 in FIG. 3. The system overview 301 shows the iVR system 222, including the hand-held controller 124 and the headset 126, and the high-resolution motion capture system 222, including the plurality of reflective markers 112 positioned on the user 101.

The plurality of virtual reality-guided movements 300 include a shoulder rotation (SR) movement 302, a side arm raise (SAR) movement 304, a forward arm raise (FAR) movement 306, an external rotation (ExR) movement 308, an abducted rotation (AbR) movement 310, a mixed press (MxdPr) movement 312, and a mixed circles (MxdCr) movement 314, such as mentioned above. For each movement, an anatomical model shows how an arm 316 moves with respect to a torso 318 to follow the butterfly 104 with the hand-held controller 124 (e.g., by placing the orb 106 on the butterfly 104, as shown to the user via the headset 126), such as described with respect to FIG. 1. Although the arm 316 is the right arm, it may be understood that similar movements may be performed with the left arm.

For the SR movement 302, the butterfly 104 moves from a position A to a position C along a path 320. The path 320 is linear and may be parallel to a ground surface, for example. Further, the path 320 is perpendicular to an axis of rotation 322. For example, the shoulder joint may rotate around a position B that is located between the position A and the position C on the path 320. For the SAR movement 304, the butterfly 104 moves from a position A, through a position B, and to a position C along a path 324. The path 324 is curved between the position A and the position C. Further, the position A is below the torso 318, while the position C is above the torso 318. The SAR movement 304 is a lateral arm movement, to the side of the torso 318, whereas the FAR movement 306 is in front of the torso 318. For the FAR movement, 306, the butterfly 104 moves from a position A, through a position B, and to a position C along a path 326. Similar to the path 324 of the SAR movement 304, the path 326 is curved, and the butterfly 104 begins below the torso 318 (e.g., at the position A) and ends above the torso 318 (e.g., at the position C). Further, for each of the SR movement 302, the SAR movement 304, and the FAR movement 306, the arm 316 is substantially straightened and unbent at the elbow.

In contrast, for each of the ExR movement 308 and the AbR movement 310, the arm 316 is bent approximately 90 degrees at the elbow. For the ExR movement 308, the butterfly 104 moves from a position A at the left side of the torso 318, through a position B, and to a position C on the right side of the torso 318 along a path 328, causing the shoulder to rotate about an axis or rotation 330. The path 328 is linear and may be parallel to the ground surface and perpendicular to the axis of rotation 330. For the AbR movement 310, the butterfly 104 moves from a position A that is below the torso 318, through a position B, and to a position C that is above the torso 318 along a path 332. The path 332 is on the right side of the torso 318 so that the arm 316 does not move across the torso 318 during the AbR movement 310. Further, the path 332 may be perpendicular to the ground surface and perpendicular to, for example, the path 328 of the ExR movement 308.

The MxdPr movement 312 includes a plurality of paths that each begin at an origin 334. The butterfly 104 moves from the origin 334 to a position A along a first path 336, from the origin 334 to a position B along a second path 338, from the origin 334 to a position C along a third path 340, from the origin 334 to a position D along a fourth path 342, and from the origin 334 to a position E along a fifth path 344. For example, the butterfly 104 may return from the position A to the origin 334 along the first path 336 before moving from the origin 334 to the position B along the second path 338, etc. until returning to the origin 334 from the position E. For example, the MxdPr movement 312 may guide the arm 316 from a bent to a substantially straightened position at a plurality of different angles (e.g., according to each of the plurality of paths).

The MxdCr movement 314 includes a plurality of circular (or elliptical) paths. For example, the butterfly 104 may move from an origin 346 along a first path 348, which sweeps from in front of the torso 318 to the back of the torso 318 before returning to the origin 346. The butterfly may then move from the origin 346 along a second path 350, which may be a substantially circular path in front of the torso 318.

Turning now to FIG. 4, a set of loss function graphs 400 demonstrate how early stopping may be used to ensure the models of the machine learning model 238 of FIG. 2 (e.g., the models shown in Table 1) do not over train. A first loss function graph 402 shows training and validation for an elevation plane angle model, a second loss function graph 404 shows training and validation for an elevation plane torque model, a third loss function graph 406 shows training and validation for a shoulder elevation angle model, a fourth loss function graph 408 shows training and validation for a shoulder elevation torque model, a fifth loss function graph 410 shows training and validation for a shoulder rotation angle model, and a sixth loss function graph 412 shows training and validation for a shoulder rotation torque model. For each of the first through sixth graphs, the horizontal axis represents a number of epochs, where each epoch corresponds to an instance of the corresponding model working through the entire training dataset, and the vertical axis represents the loss, which measures how far the estimated value (e.g., determined via the model) is from its true value (e.g., determined from the biomechanical simulation 214 of FIG. 2). Further, for each of the first through sixth graphs, training data is shown by a solid plot, while validation data is shown by a dashed plot, as indicated by a legend 414.

As shown in FIG. 4, the loss does not substantially decrease for the training data or the validation data for any of the six models after around 10-15 epochs. As such, the training may be terminated once the corresponding model does not improve, as indicated by a further decrease in the loss, after 5 epochs. As an illustrative example, referring to the first loss function graph 402, the training plot for the elevation plane angle model shows the loss function reacting a minimum at around 10 epochs. Thus, the training for the elevation plane angle model may be terminated at around 15 epochs. Referring to the second loss function graph 404, the training plot for the elevation plane torque model reaches a minimum at around 15 epochs. Thus, the training for the elevation plane torque model may be terminated at around 20 epochs. By preventing overfitting, the six models may be able to more accurately evaluate new data by not, for example, evaluating the new data based on learned details of the noise and random fluctuations in the training data.

The vertical displacement of the hand-held controller 124 introduced in FIG. 1 during gameplay is shown as a set of graphs 500 in FIG. 5. That is, the set of graphs 500 shows the vertical displacement (vertical axis, in meters) over time (horizontal axis, in seconds) for a plurality of users and game trials for each movement described with respect to FIG. 3. Data for the FAR movement is shown in a first plot 502, data for the SAR movement is shown in a second plot 504, data for the SR movement is shown in a third plot 506, data for the ExR movement is shown in a fourth plot 508, data for the AbR movement is shown in a fifth plot 510, data for the MxdPr movement is shown in a sixth plot 512, and data for the MxdCr movement is shown in a seventh plot 514.

As can be seen in FIG. 5, the different movements have varying uniformity among users. For example, the first plot 502 and the second plot 504 show that the FAR movement and the SAR movement have high uniformity between users and game trials, as indicated by the high overlap of the different traces. In contrast, the ExR, AbR, MxdPr, and MxdCr movements have relatively low uniformity, as indicated by the variability in the traces for the fourth plot 508, the fifth plot 510, the sixth plot 512, and the seventh plot 514. However, even with the lower uniformity of motion, the machine learning model 238 of FIG. 2 is still able to accurately predict the corresponding joint angles and joint torques during each motion.

This is exemplified in FIG. 6, which shows example graphs 600 comparing shoulder elevation angle and shoulder elevation torque determined from the biomechanical simulation 214 of FIG. 2 with predictions from the machine learning model 238 of FIG. 2 for the FAR movement. In particular, the shoulder elevation angle is shown in a first graph 602, where the vertical axis represents the shoulder elevation angle (in degrees) and the horizontal axis represents time (in seconds). The shoulder elevation torque is shown in a second graph 604, where the vertical axis represents the shoulder elevation torque (in Nm) and the horizontal axis represents time (in seconds). For each of the first graph 602 and the second graph 604, results from the offline method using the biomechanical simulation are shown in red, whereas the predictions from the machine learning-based method are shown in green, as indicated by a legend 606.

As shown in the first graph 602, the shoulder elevation angles predicted using the machine learning model and data gathered via an iVR system (e.g., the iVR system 222 of FIG. 2) during the FAR movement closely agrees with those determined using the biomechanical simulation and data gathered via an optical tracking system (e.g., the high-resolution motion capture system 208 of FIG. 2). Similarly, the shoulder elevation torques predicted using the machine learning model and data gathered via the iVR system during the FAR movement also closely agree with the shoulder elevation torques determined via the biomechanical simulation and data gathered with the optical tracking system during the FAR movement. As such, the machine learning model provides highly accurate predictions for the shoulder elevation angles and the shoulder elevation torques during the FAR movement. Although the FAR movement is shown as an example, it may be understood that the data may be similar for the other movements and shoulder parameter models described herein.

In this way, an off-the-shelf iVR system paired with machine learning may accurately provide predictive kinematics for evaluating rehabilitative exercises. As a result, the iVR system may be utilized for telehealth, thereby alleviating the loss of in-person evaluation methods through remote estimation of range-of-motion and joint torques. Accurate and consistent measurement of range-of-motion is fundamental to monitoring recovery during physical therapy, and measuring upper limb kinematics is one of the most challenging problems in human motion estimation. Because the shoulder cannot be estimated by simple single plane joint models, the present disclosure addresses this complex problem with a low-cost solution that can be used both in a clinic and at a patient's home. The present disclosure illustrates that off-the-shelf iVR headsets can be employed for motion analysis in comparison to complex and expensive optical motion capture methods, which rely on expensive equipment and accurate placement on anatomical landmarks. By providing a low cost, easy to use, and accurate system for remote rehabilitation, patients may provide more frequent measurements from their homes, enabling therapists to have a more detailed remote patient analysis in guiding physical rehabilitation. Overall, patients may be empowered by being able to complete at-home guided exercises at a time that works with their schedule over a longer duration. As a result, positive recovery outcomes may be increased.

The technical effect of using a machine learning model to predict joint kinematics during guided exercises based on data acquired with an immersive virtual reality system, the machine learning model trained based on data acquired via an optical motion tracking system, is that physical rehabilitation may be accurately monitored via telehealth.

The disclosure also provides support for a system, comprising: an immersive virtual reality (iVR) system, the iVR system including a headset and a hand-held controller, and machine readable instructions executable to: predict joint kinematics using a machine learning model based on motion data received from the iVR system during gameplay of a virtual reality-guided exercise with the iVR system. In a first example of the system, the machine learning model is trained using joint angles and joint torques determined via biomechanical simulation using data obtained via an optical motion tracking system. In a second example of the system, optionally including the first example, the machine learning model comprises a plurality of separate models for different parameters of the joint kinematics. In a third example of the system, optionally including one or both of the first and second examples, the plurality of separate models for the different parameters of the joint kinematics comprise one or more of an elevation plane angle model, an elevation angle model, an elevation plane torque model, an elevation torque model, and a rotation torque model. In a fourth example of the system, optionally including one or more or each of the first through third examples, the machine learning model is trained using an extreme gradient boost algorithm, an artificial neural network, a convolutional neural network, a long short-term memory, and/or a random forest.

The disclosure also provides support for a method, comprising: training a machine learning model using biomechanical simulation parameters generated using data from a high-resolution motion capture system, and predicting joint parameters via the machine learning model by inputting data from a low-resolution motion capture system into the machine learning model. In a first example of the method, the low-resolution motion capture system includes an immersive virtual reality (iVR) headset and a hand-held controller, and where predicting the joint parameters via the machine learning model by inputting data from the low-resolution motion capture system into the machine learning model comprises inputting a rotation and a position of the hand-held controller into the machine learning model. In a second example of the method, optionally including the first example, the data from the high-resolution motion capture system and the data from the low-resolution motion capture system are both obtained during a series of exercises guided by a game displayed via the iVR headset. In a third example of the method, optionally including one or both of the first and second examples, the joint parameters comprise a shoulder joint torque and a shoulder joint angle. In a fourth example of the method, optionally including one or more or each of the first through third examples, training the machine learning model comprises training the machine learning model using an extreme gradient boost algorithm.

The following claims particularly point out certain combinations and sub-combinations regarded as novel and non-obvious. These claims may refer to “an” element or “a first” element or the equivalent thereof. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, also are regarded as included within the subject matter of the present disclosure.

1. A system, comprising:
an immersive virtual reality (iVR) system, the iVR system including a headset and a hand-held controller; and
machine readable instructions executable to:
predict joint kinematics using a machine learning model based on motion data received from the iVR system during gameplay of a virtual reality-guided exercise with the iVR system.

2. The system of claim 1, wherein the machine learning model is trained using joint angles and joint torques determined via biomechanical simulation using data obtained via an optical motion tracking system.

3. The system of claim 1, wherein the machine learning model comprises a plurality of separate models for different parameters of the joint kinematics.

4. The system of claim 3, wherein the plurality of separate models for the different parameters of the joint kinematics comprise one or more of an elevation plane angle model, an elevation angle model, an elevation plane torque model, an elevation torque model, and a rotation torque model.

5. The system of claim 1, wherein the machine learning model is trained using an extreme gradient boost algorithm, an artificial neural network, a convolutional neural network, a long short-term memory, and/or a random forest.

6. A method, comprising:
training a machine learning model using biomechanical simulation parameters generated using data from a high-resolution motion capture system; and
predicting joint parameters via the machine learning model by inputting data from a low-resolution motion capture system into the machine learning model.

7. The method of claim 6, wherein the low-resolution motion capture system includes an immersive virtual reality (iVR) headset and a hand-held controller, and where predicting the joint parameters via the machine learning model by inputting data from the low-resolution motion capture system into the machine learning model comprises inputting a rotation and a position of the hand-held controller into the machine learning model.

8. The method of claim 7, wherein the data from the high-resolution motion capture system and the data from the low-resolution motion capture system are both obtained during a series of exercises guided by a game displayed via the iVR headset.

9. The method of claim 6, wherein the joint parameters comprise a shoulder joint torque and a shoulder joint angle.

10. The method of claim 6, wherein training the machine learning model comprises training the machine learning model using an extreme gradient boost algorithm.
